(19) United States
(12) Patent Application Publication
(10) Pub. No.: US 2005/0019285 A1
(43) Pub. Date: Jan. 27, 2005

(54) Cosmetic composition of foundation type for making up dark skins

(75) Inventors: Honjung Lee, Paris (FR); Nicolas Soistier, Le Kremlin-Bicetre (FR); Jean-Christophe Simon, Alsbach-Hahnlein (DE)

Correspondence Address: OLIFF & BERRIDGE, PLC, P.O. BOX 19928, ALEXANDRIA, VA, 22320, US

(73) Assignee: L'Oreal, Paris (FR)

(21) Appl. No.: 10/743,523

(22) Filed: Dec. 23, 2003

Related U.S. Application Data
(60) Provisional application No. 60/439,803, filed on Jan. 14, 2003.

(30) Foreign Application Priority Data
Dec. 24, 2002 (FR) 02 116655

(51) Int. Cl. A61K 7/021

(52) U.S. Cl. 424/63

(57) ABSTRACT

The present invention relates to a composition of foundation type, especially for making up the skin, in particular dark skins, comprising, in a physiologically acceptable medium, at least one colouring agent and reflective particles, the said composition having a hue in the colour range extending from pink-beige to orange-brown.

This application claims the benefit of U.S. Provisional Application No. 60/439,803 filed on Jan. 14, 2003, the disclosure of which is incorporated by reference herein.

### BACKGROUND OF THE INVENTION

The present invention relates to cosmetic compositions to be applied to the skin, and more particularly compositions of foundation type for dark skins, including black and mixed-race skins.

For the purposes of the present invention, the expression “composition of foundation type” denotes a composition for making up human skin. In the present case, it may be a foundation to be applied to the face or the neck, a concealer product, a complexion corrector, a tinted cream or makeup base for the face or a makeup composition for the body.

In general, compositions of this type contain, besides a fatty phase such as wax or oil, mineral or organic fillers and coloring agents.

The coloring agents are used as opacifiers and/or dyes and are present in a concentration that is sufficient to give the desired color.

As regards the fillers, they are conventionally used to modify the rheology, to adjust the texture of the composition, to reduce its sheen and/or as a mattifying agent to hide skin defects on the surface to be made up.

The fillers usually used are colorless or white, mineral or synthetic particles of any form, which are insoluble in the medium of the composition irrespective of the temperature at which the composition is manufactured. They are generally silica, mica, talc, kaolin and more particularly titanium oxides. These fillers are conventionally used in a proportion of from 1% to 80% and especially from 1% to 50% by weight relative to the total weight of the cosmetic composition.

However, in the case of compositions of foundation type more particularly intended for “dark” skins and especially black skin, the presence of these white particles may be detrimental to the desired aesthetic effect. Specifically, these white particles may, under certain circumstances, especially associated with their proportion in the composition and/or with the color of the skin, give rise to a greyish colored effect on the made-up skin. For obvious aesthetic reasons, such an effect is particularly undesirable.

### SUMMARY OF THE INVENTION

The present invention is specifically directed towards proposing compositions of foundation type, especially for making up the skin, which are free of this side effect and which are more particularly suitable for making up dark skins and in particular black or mixed-race skins.

In particular, the present invention is directed towards proposing a composition of foundation type for dark skins, which advantageously affords, in combination, uniformity of coloration between the various areas of the face, especially the forehead, the bags under the eyes and the cheekbones, and a lightening effect that is virtually or even totally free of a greyish effect. This results in a more natural makeup effect on the made-up individual.

More specifically, according to one of its first aspects, the present invention relates to a composition of foundation type especially for making up the skin, in particular dark skins, comprising, in a physiologically acceptable medium, at least one coloring agent and reflective particles, the said composition having a hue in the color range extending from pink-beige to orange-brown.

According to another of its aspects, the present invention relates to a composition of foundation type, especially for making up the skin, in particular dark skins, comprising, in a physiologically acceptable medium, at least one coloring agent and reflective particles, the said composition having a hue angle h ranging from 40° to 70° and especially from 50° to 70°, and a saturation C* ranging from 20 to 50.

According to another of its aspects, the present invention relates to a composition of foundation type, especially for making up the skin, in particular dark skins, comprising, in a physiologically acceptable medium, at least one coloring agent and reflective particles, the said composition having a reflectance ranging from 10% to 45% in the range from 600 to 680 nm.

According to another of its aspects, the present invention relates to a method for making up a dark skin, characterized in that it comprises the application to the skin of at least one composition of foundation type in accordance with the invention.

According to another of its aspects, the present invention relates to a product of foundation type especially for making up the skin, in particular dark skins, comprising at least one first and one second composition, each in a container, the first composition comprising, in a first physiologically acceptable medium, at least one coloring agent, and the second composition comprising, in a second physiologically acceptable medium, at least reflective particles.

According to another of its aspects, a subject of the present invention is also a method for making up a dark skin, comprising the application to the skin of a product in accordance with the present invention. More specifically, this method comprises the application of a first coat of one of the two compositions of the said product, also known as the base composition, followed by the application over at least a portion of the said first coat of a second coat of the other composition of the said product, also known as the surface composition.

In the present case, the makeup thus obtained is a two-coat makeup. The order of superposition and/or the mode of superposition of the two compositions, i.e. total or partial, may moreover be advantageous for giving additional aesthetic effects.

According to another of its aspects, the present invention is also directed towards the use of a composition or a product in accordance with the invention to obtain a lightening makeup result on a dark skin, especially black or mixed-race skin.

The inventors have thus found that it is possible to develop a cosmetic composition of foundation type that is free of an undesirable colored effect, by combining at least one coloring agent with reflective particles, used especially in partial or total replacement for conventional fillers.

These compositions of foundation type are found to be particularly advantageous for making up and, where appropriate, lightening dark skins.

The term “dark skins” denotes skins whose mean lightness L* measured on the forehead, the cheekbones and the chin, in the CIE 1976 calorimetric space, is less than 60 and especially less than 55. The saturation C* may be, for example, between 8 and 30, especially between 10 and 30 or even between 12 and 28. The hue angle values h may be, for example, between about 38° and about 77° and especially between 46° and 63°, for example between 46° and 54°. The lightness values L* may be less than or equal to 50, or even 45 or 40 for the darkest skins, while at the same time remaining above 30 for the majority of skins. Dark skins are encountered, for example, among the African, Afro-American, Hispano-American, Indian and Maghrebine populations.

In general, the compositions of foundation type according to the invention contain a coloring agent or a mixture of coloring agents in an amount that is sufficient to give them a hue in the color range extending from pink-beige to orange-brown, passing especially through orange-yellow.

The composition of foundation type according to the invention may also be characterized by its hue angle h and its saturation C*.

In the present case, the composition of foundation type according to the invention advantageously has a hue angle h ranging from 40° to 70° and especially from 50° to 70°, and a saturation C* of from 20 to 50. These parameters are characterized directly on the composition according to the protocol presented below in the examples.

The composition of foundation type according to the invention may also be characterized by its reflectance spectrum. Thus, for the range from 600 to 680 nm, the reflectance ranges from 10% to 45% and more specifically from 12% to 40%.

Moreover, this reflectance may be less than 20% in the range from 450 nm to 500 nm.

This combination of characteristics may specifically be obtained, in the context of the present invention, by means of a combination of specific coloring agents and of reflective particles.

### DESCRIPTION OF THE PREFERRED EMBODIMENTS

Coloring Agents

Coloring agents that are suitable for the invention can produce, alone or as a mixture, a yellow or orange coloration. In other words, they have a significant reflectance in the range from 550 to 675 nm.

The coloring agent(s) may be present in the composition of foundation type, especially the base or surface composition of the product of foundation type according to the invention, in a content ranging from 0.5% to 30% by weight, especially ranging from 2% to 20% by weight and in particular from 5 to 18% by weight relative to the total weight of the composition under consideration.

The coloring agent(s) may be chosen from mineral or organic pigments, coloring polymers, water-soluble or liposoluble dyes, organic lacquers, metallic powders, and mixtures thereof. They may be chosen especially from those mentioned in the CTFA Cosmetic Ingredient Handbook, 3rd Edition Cosmetic and Fragrance Association., Inc., Washington D.C. (1982).

As nonlimiting illustrations of mineral coloring agents, mention may be made more particularly of yellow, red and brown metal oxides, for instance iron oxides.

As metallic powders, mention may be made of copper powder.

The pigments FDC Yellow No. 5 (disodium salt of tartrazine) are suitable especially as organic pigments.

As illustrations of organic lacquers that are suitable for the invention, mention may be made more particularly of FDC Yellow No. 5 and No. 6 Al Lake.

The water-soluble dyes may be chosen, for example, from the brown dye identified by the name “caramel” according to the Color Index; the yellow dyes identified by the Color Index numbers 10316, 13015, 18690, 18820, 18965, 19140, 45430, 47005, 75100 and that known as Lactoflavin; the orange dyes identified by the Color Index numbers 14270, 15510, 15980, 15985, 16230, 20170, 40215; the red dyes identified by the Color Index numbers 14700, 14720, 14815, 15620, 16035, 16185, 16255, 16290, 17200, 18050, 18130, 18736, 24790, 27290, 45100, 45220, 45380, 45405, 45410, 45425, 45430, 75470, and mixtures thereof.

The liposoluble dyes may be chosen, for example, from the brown dye identified by the Color Index number 12010; the yellow dyes identified, respectively, by the Color Index numbers 12700, 21230, 47000, 75125, 75135; the orange dyes identified by the Color Index numbers 11920, 40800, 40820, 40825, 40850, 45396, 75120, 75130 and capasanthine and the red dye identified by number 12150, and mixtures thereof.

The dyeing polymer is a polymer comprising at least one organic dye group. The dye group may be grafted, especially via covalent bonding, onto the chain of the polymer. The dye polymer generally contains less than 10% by weight of dyestuff relative to the total weight of the polymer.

This dye polymer may be of any chemical nature, especially polyester, polyamide, polyurethane, polyacrylic, poly(meth)acrylic, polycarbonate, of natural origin, for instance cellulose or chitosan polymers, or a mixture thereof, and in particular a polyester or polyurethane.

In particular, the dye polymer may be a copolymer based on at least two different monomers, at least one of which is an organic dye monomer.

Such dye polymers are described especially in patents or patent applications U.S. Pat. No. 5,032,670, U.S. Pat. No. 4,999,418, U.S. Pat. No. 5,106,942, U.S. Pat. No. 5,030,708, U.S. Pat. No. 5,102,980, U.S. Pat. No. 5,043,376, U.S. Pat. No. 5,104,913, U.S. Pat. No. 5,281,659, U.S. Pat. No. 5,194,463, U.S. Pat. No. 4,804,719, WO 92/07913 and EP-A-747036.

As illustrations of monomers for known dye polymers, mention may be made especially of anthraquinones, methines, bis-methines, azamethines, arylidenes, 3H-dibenzo[7,i-j]isoquinolines, 2,5-diarylaminoterephthalic acids and esters thereof, phthaloylphenothiazines, phthaloylphenoxazines, phthaloylacridone, anthrapyrimidines, anthrapyrazoles, phthalocyanins, quinophthalones, indophenols, perinones, nitroarylamines, benzodifuran, 2H-1-benzopyran-2-one, quinophthalones, perylenes, quinacridones, triphenodioxazines, fluoridines, 4-amino-1,8-naphthalimides, thioxanthrones, benzanthrones, indanthrones, indigo, thioindigo, xanthene, acridine, azine and oxazine.

Needless to say, a person skilled in the art is capable, via his general knowledge, of selecting the monomers to adjust the desired color effect according to the invention.

The color agent(s) and especially the pigments used in the context of the present invention may be used either in their crude form or in a pretreated form, especially pretreated at the surface thereof. The aim of this treatment is generally to increase the stability of the color and to facilitate their incorporation into cosmetic formulations. In particular, coloring agents treated in order to make them hydrophobic will be more readily dispersible in an oily phase.

An illustration of these surface treatments that may be mentioned especially is the treatment consisting in treating the pigment with a hydrophobic and oil-repellant agent of perfluoroalkyl phosphate derivative type, as described in EP 1 086 683.

Similarly, it may be useful to treat the coloring agents, and especially the pigments, with a material that makes them compatible with the oily phases and especially the silicone phases used in cosmetic formulations. Pigments of this type are described especially in patent U.S. Pat. No. 5,143,722.

According to one particular embodiment, the coloring agents used according to the invention are of yellow, orange, brown or red color.

As illustrations of coloring agents that are more particularly suitable for the invention, mention may be made especially of brown iron oxide and yellow iron oxide, coated with perfluoroalkyl phosphate, and titanium oxide treated with alumina, coated with perfluoroalkyl phosphate, or titanium oxide coated with perfluoroalkyl phosphate, for instance, in particular, the pigmentary pastes sold under the trade names Yellow Iron Oxide Covafluor, PF5 Yellow 601 (yellow) and PF5 Red R516L (red), PF5 Black BL100 by the company Daito, under the trade names FA50DRF, FA50DYF, FA65DF and FA65DBF by the company Kobo, ultramarine blue coated with perfluoroalkyl phosphate, under the trade name PF5 Ultramarine No. 801 by the company Daito, the disodium salts of tartrazine and the aluminium lakes of Allura red on alumina sold by the company Noveon under the names FDC Yellow No. 6, Al Lake and FDC Yellow No. 5 Al Lake, and mixtures thereof.

Reflective Particles

For the purposes of the present invention, the term “reflective particles” denotes particles whose size, structure, especially the thickness of the layer(s) of which they are composed and their physical and chemical natures, and surface state allow them to reflect incident light. This reflection may, where appropriate, have an intensity sufficient to create at the surface of the composition of foundation type according to the invention, when this composition is applied to the support to be made up, highlight points that are visible to the naked eye, i.e. more luminous points which contrast with their environment and appear to shine.

The reflective particles are also selected so as not to significantly impair the coloring effect generated by the coloring agents combined therewith and more particularly so as to optimize this effect in terms of color rendition. They may more particularly have a yellow, pink, red, bronze, orange, brown, gold and/or coppery color or glint.

The reflective particles may be present in the composition according to the invention and the base or surface composition of the product according to the invention in a content ranging from 0.5% to 60% relative to the total weight of the composition, especially from 1% to 30% by weight, in particular from 2% to 20% by weight or even from 3% to 10% by weight.

These particles may have varied forms. These particles may especially be in the form of platelets or in globular form, in particular in spherical form.

Whatever their form, the reflective particles may have a multilayer or non-multilayer structure and, in the case of a multilayer structure, for example at least one layer of uniform thickness, especially of a reflective material.

When the reflective particles do not have a multilayer structure, they may be composed, for example, of metal oxides, for example titanium oxide or iron oxide obtained synthetically.

When the reflective particles have a multilayer structure, they may, for example, comprise a natural or synthetic substrate, especially a synthetic substrate at least partially coated with at least one layer of a reflective material especially of at least one metal or metallic compound. The substrate may be monomaterial or multimaterial, and organic and/or mineral.

More particularly, it may be chosen from glasses, ceramics, graphite, metal oxides, aluminas, silicas, silicates, especially aluminosilicates and borosilicates, synthetic mica and mixtures thereof, this list not being limiting.

The reflective material may comprise a layer of metal or of a metallic compound.

Glass particles coated with a metallic layer are described especially in documents JP-A-09 188 830, JP-A-10 158 450, JP-A-10 158 541, JP-A-07 258 460 and JP-A-05 017 710.

Again as examples of reflective particles comprising a mineral substrate coated with a layer of metal, mention may also be made of particles comprising a borosilicate substrate coated with silver, also known as “white nacres”.

Particles containing a glass substrate coated with silver, in the form of platelets, are sold under the name Microglass Metashine REFSX 2025 PS by the company Toyal. Particles containing a glass substrate coated with a nickel/chromium/molybdenum alloy are sold under the name Crystal Star GF 550, GF 2525 by this same company.

Irrespective of their form, the reflective particles may also be chosen from particles containing a synthetic substrate coated at least partially with at least one layer of at least one metallic compound, especially a metal oxide chosen, for example, from titanium oxide, especially TiO₂, iron oxide, especially Fe₂O₃, tin oxide, chromium oxide, barium sulphate and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂ and mixtures or alloys thereof.

As examples of such particles, mention may be made, for example, of particles comprising a synthetic mica substrate coated with titanium dioxide, or glass particles coated either with brown iron oxide, titanium oxide, tin oxide or with one of the mixtures thereof, such as those sold under the brand name Reflecks® by the company Engelhard.

The reflective particles may or may not be goniochromatic and/or may or may not be interference particles. For the purposes of the invention, they comprise nacres and goniochromatic coloring agents.

The term “nacres” should be understood as meaning colored particles of any form, which may or may not be iridescent, produced especially by certain molluscs in their shell or else synthesized, and which have a color effect by optical interference.

The nacres may be chosen from nacreous pigments such as titanium mica coated with an iron oxide, mica coated with bismuth oxychloride, titanium mica coated with chromium oxide, titanium mica coated with an organic dye, especially of the abovementioned type, and also nacreous pigments based on bismuth oxychloride. They may also be mica particles at the surface of which are superposed at least two successive layers of metal oxides and/or of organic dyestuffs.

The nacres may more particularly have a yellow, pink, red, bronze, orange, brown, gold and/or coppery color or glint.

As illustrations of nacres that may be used in the context of the present invention, mention may be made especially of the gold-colored nacres sold especially by the company Engelhard under the name Brillant Gold 212G (Timica), Gold 222C (Cloisonne), Sparkle Gold (Timica), Gold 4504 (Chromalite) and Monarch Gold 233X (Cloisonne); the bronze nacres sold especially by the company Merck under the name Bronze Fine (17384) (Colourona) and Bronze (17353) (Colourona) and by the company Engelhard under the name Super Bronze (Cloisonne); the orange nacres sold especially by the company Engelhard under the name Orange 363C (Cloisonne) and Orange MCR 101 (Cosmica) and by the company Merck under the name Passion Orange (Colourona) and Matte Orange (17449) (Microna); the brown nacres sold especially by the company Engelhard under the name Nu-antique Copper 340XB (Cloisonne) and Brown CL4509 (Chromalite); the nacres with a copper glint sold especially by the company Engelhard under the name Copper 340A (Timica); the nacres with a red glint sold especially by the company Merck under the name Sienna Fine (17386) (Colourona); the nacres with a yellow glint sold especially by the company Engelhard under the name Yellow (4502) (Chromalite); the red nacres with a golden glint sold especially by the company Engelhard under the name Sunstone G012 (Gemtone); the pink nacres sold especially by the company Engelhard under the name Tan Opale G005 (Gemtone); the black nacres with a golden glint sold especially by the company Engelhard under the name Nu-antique Bronze 240 AB (Timica), the blue nacres sold especially by the company Merck under the name Matte Blue (17433) (Microna), the white nacres with a silvery glint sold especially by the company Merck under the name Xirona Silver, and the golden green pink-orange nacres sold especially by the company Merck under the name Indian Summer (Xirona), and mixtures thereof.

It may also be envisaged to use a goniochromatic coloring agent as reflective particles, with the proviso that this agent satisfies the hue effect stipulation required according to the invention and does not otherwise disrupt the visual perception of the composition in terms of the color effect. This goniochromatic coloring agent may be chosen especially from multilayer interference structures.

Fillers

As mentioned previously, the presence of reflective particles in a proportion sufficient to ensure a filler function makes it possible to significantly reduce the amount, or even to avoid the presence, of conventional filler(s) of white particle type.

In the present case, the compositions of foundation type in accordance with the invention may contain less than 5% or even 3% by weight of white particles and especially of titanium oxide, or may even be free of titanium oxide.

Needless to say, it nevertheless remains possible to combine the reflective particles according to the invention with one or more other conventional fillers, with the proviso that these conventional fillers are used in an amount such that they do not affect the aesthetic effect desired by the compositions of foundation type claimed, i.e. they do not excessively impart a greyish appearance to the made-up skin when this skin is coated with a composition in accordance with the invention. A person skilled in the art is capable, by means of his knowledge, of making this adjustment.

These fillers may be mineral or organic and of any form: platelet, spherical or oblong, irrespective of the crystallographic form (for example leaflet, cubic, hexagonal, orthorhombic, etc.). Mention may be made of talc, mica, silica, kaolin, polyamide powder (Nylon®) (Orgasol® from Atochem), poly-β-alanine powder and polyethylene powder, tetrafluoroethylene polymer powders (Teflon®), lauroyllysine, starch, boron nitride, hollow polymer microspheres such as those made of polyvinylidene chloride/acrylonitrile, for instance Expancel® (Nobel Industrie), made of acrylic acid copolymers (Polytrap® from the company Dow Corning) or made of polymethyl methacrylate (Covabead from Wackherr), silicone resin microbeads (for example Tospearls® from Toshiba), polyorganosiloxane elastomer particles, precipitated calcium carbonate, magnesium carbonate, magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and preferably from 12 to 18 carbon atoms, for example zinc, magnesium or lithium stearate, zinc laurate or magnesium myristate, and mixtures thereof.

It may be advantageous to favour the choice of transparent complementary fillers, for instance fumed silica.

This or these filler(s) may be present in a proportion of from 0.1% to 20% by weight, preferably 2% to 15% by weight and better still from 2% to 10% by weight relative to the total weight of the composition of foundation type, especially of the base or surface compositions of the product of foundation type.

Physiologically Acceptable Medium

The term “physiologically acceptable medium” denotes a non-toxic medium that may be applied to human skin. The physiologically acceptable medium is generally suited to the nature of the skin onto which the composition of foundation type is to be applied and also to the form in which the composition is intended to be packaged, especially fluid at room temperature and at atmospheric pressure.

As mentioned previously, the compositions of foundation type according to the invention, especially the base and/or surface compositions of the product of foundation type according to the invention, may be formulated in a fluid or solid form of free, compact or cast powder type.

They may especially be, independently of each other, in an anhydrous form or in the form of a gel, of direct, inverse or multiple emulsion combining at least one aqueous phase and at least one fatty phase.

Aqueous Phase

The composition of foundation type according to the invention, especially the base composition and/or the surface composition of the product of foundation type according to the invention, may comprise at least one aqueous medium, constituting an aqueous phase, which may form the continuous phase of the composition of foundation type under consideration.

The aqueous phase may consist essentially of water.

It may also comprise a mixture of water and of water-miscible organic solvent (miscible in water to greater than 50% by weight at 25° C.), for instance lower monoalcohols containing from 1 to 5 carbon atoms, such as ethanol, isopropanol, glycols containing from 2 to 8 carbon atoms, such as propylene glycol, ethylene glycol, 1,3-butylene glycol, dipropylene glycol, C₃-C₄ ketones and C₂-C₄ aldehydes.

The aqueous phase (water and optionally the water-miscible organic solvent) may be present in a content ranging from 1% to 95% by weight, especially ranging from 3% to 80% by weight, and in particular ranging from 5% to 60%, by weight relative to the total weight of the composition under consideration.

Such a medium may also comprise a volatile oil as defined below.

Fatty Phase

The composition of foundation type according to the invention, especially the base composition and/or the surface composition of the product of foundation type according to the invention, may comprise a fatty phase and especially at least one fatty substance that is liquid at room temperature (25° C.) and/or a fatty substance that is solid at room temperature, such as waxes, pasty fatty substances and gums, and mixtures thereof. The fatty phase may also contain lipophilic organic solvents.

The composition of foundation type may contain, for example, a continuous fatty phase, which may contain less than 5% water, especially less than 1% water, relative to its total weight, and may in particular be in anhydrous form.

The fatty phase of the composition according to the invention may especially comprise, as liquid fatty substance, at least one volatile or non-volatile oil or a mixture thereof.

For the purposes of the invention, the term “volatile oil” means any oil capable of evaporating on contact with the skin in less than one hour, at room temperature and atmospheric pressure. The volatile oils of the invention are volatile cosmetic oils, which are liquid at room temperature, having a non-zero vapour pressure, at room temperature and atmospheric pressure, ranging in particular from 0.01 to 300 mmHg (1.33 Pa to 40 000 Pa) and preferably greater than 0.3 mmHg (30 Pa).

The term “non-volatile oil” means an oil that remains on the skin at room temperature and atmospheric pressure for at least several hours and that especially has a vapour pressure of less than 0.01 mmHg (1.33 Pa).

These volatile or non-volatile oils may be hydrocarbon-based oils, silicone oils or mixtures thereof. The term “hydrocarbon-based oil” means an oil mainly containing hydrogen and carbon atoms and possibly oxygen, nitrogen, sulphur and phosphorus atoms.

The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane, isohexadecane, and for example the oils sold under the trade names Isopars® or Permetyls®, branched C₈-C₁₆ esters such isohexyl neopentanoate, and mixtures thereof. Other volatile hydrocarbon-based oils, for instance petroleum distillates, especially those sold under the name Shell Solt® by the company Shell, may also be used.

Volatile oils that may also be used include volatile silicones, for instance volatile linear or cyclic silicone oils, especially those with a viscosity≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethyl-cyclohexasiloxane, heptamethylhexyltrisiloxane, heptamethyloctyltrisiloxane, hexamethyldisiloxane, octamethyltrisiloxane, decamethyltetrasiloxane and dodecamethyl-pentasiloxane, and mixtures thereof.

The volatile oil may be present in a composition according to the invention in a content ranging from 0.1% to 98% by weight, especially from 1% to 65% by weight, and in particular from 2% to 50% by weight, relative to the total weight of the composition.

The non-volatile oils may be chosen especially from non-volatile fluoro and/or silicone hydrocarbon-based oils.

Non-volatile hydrocarbon-based oils that may especially be mentioned include:

hydrocarbon-based oils of animal origin,

hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially wheatgerm oil, sunflower oil, grapeseed oil, sesame seed oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stearineries Dubois or those sold under the names Miglyol 810, 812 and 818® by the company Dynamit Nobel,

synthetic ethers containing from 10 to 40 carbon atoms,

linear or branched hydrocarbons of mineral or synthetic origin, such as petroleum jelly, polydecenes, hydrogenated polyisobutene such as parleam, and squalane, and mixtures thereof,

synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol heptanoates, octanoates, decanoates or ricinoleates, for instance propylene glycol dioctanoate; hydroxylated esters, for instance isostearyl lactate or diisostearyl malate; polyol esters and pentaerythritol esters,

fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol,

higher fatty acids such as oleic acid, linoleic acid or linolenic acid, and mixtures thereof.

The non-volatile silicone oils that may be used in the composition according to the invention may be non-volatile polydimethylsiloxanes (PDMS), polydimethylsiloxanes comprising alkyl or alkoxy groups, which are pendant and/or at the end of a silicone chain, these groups each containing from 2 to 24 carbon atoms, phenylsilicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates.

The non-volatile oils may be present in a composition according to the invention in a content ranging from 0.01% to 90% by weight, especially from 0.1% to 85% by weight and in particular from 1% to 70% by weight relative to the total weight of the composition.

More generally, the liquid fatty substance may be present in a proportion of from 0.01% to 90% by weight and especially from 0.1% to 85% by weight relative to the weight of the fatty phase.

regards the fatty substance that is solid at room temperature and atmospheric pressure, it may be chosen from waxes, pasty fatty substances and gums, and mixtures thereof. This solid fatty substance may be present in a proportion of from 0.01% to 50%, especially from 0.1% to 40% and in particular from 0.2% to 30% by weight relative to the total weight of the fatty phase.

Thus, a composition according to the invention may comprise at least one fatty substance that is pasty at room temperature.

For the purposes of the invention, the term “pasty fatty substance” means fatty substances with a melting point ranging from 20 to 55° C. and preferably 25 to 45° C., and/or a viscosity at 40° C. ranging from 0.1 to 40 Pa.s (1 to 400 poises) and preferably 0.5 to 25 Pa.s, measured using a Contraves TV or Rheomat 80 viscometer, equipped with a spindle rotating at 60 Hz. A person skilled in the art can select the spindle for measuring the viscosity from the spindles MS-r3 and MS-r4, on the basis of his general knowledge, so as to be able to perform the measurement on the pasty compound tested.

Preferably, these fatty substances are hydrocarbon-based compounds optionally of polymeric type; they may also be chosen from silicone compounds; they may also be in the form of a mixture of hydrocarbon-based and/or silicone compounds. In the case of a mixture of different pasty fatty substances, the hydrocarbon-based pasty compounds (mainly containing carbon and hydrogen atoms and optionally ester groups) are preferably used, in majority proportion.

Among the pasty compounds that may be used in the composition of foundation type according to the invention, mention may be made of lanolins and lanolin derivatives, for instance acetylated lanolins, oxypropylenated lanolins or isopropyl lanolate, with a viscosity of from 18 to 21 Pa.s and preferably 19 to 20.5 Pa.s, and/or a melting point of from 30 to 55° C., and mixtures thereof. Esters of fatty acids or of fatty alcohols, especially those containing 20 to 65 carbon atoms (melting point from about 20 to 35° C. and/or viscosity at 40° C. ranging from 0.1 to 40 Pa.s) may also be used, for instance triisostearyl citrate or cetyl citrate; arachidyl propionate; polyvinyl laurate; cholesterol esters, for instance triglycerides of plant origin such as hydrogenated plant oils, viscous polyesters, for instance poly(12-hydroxystearic acid), and mixtures thereof. Triglycerides of plant origin that may be used include hydrogenated castor oil derivatives, such as “Thixinr” from Rheox.

Mention may also be made of silicone pasty fatty substances such as polydimethylsiloxanes (PDMS) of high molecular weight and in particular those with pendant chains of the alkyl or alkoxy type containing from 8 to 24 carbon atoms, and a melting point of 20-55° C., for instance stearyl dimethicones, especially those sold by the company Dow Corning under the trade names DC2503® and DC25514®, and mixtures thereof.

The pasty fatty substance may be present in a composition according to the invention in a content ranging from 0.01% to 50% by weight, preferably ranging from 0.1% to 45% by weight and better still ranging from 0.2% to 30% by weight relative to the total weight of the said composition.

The composition of foundation type according to the invention, especially the base composition and/or the surface composition of the product of foundation type according to the invention, may also comprise a wax. The wax may be solid at room temperature (25° C.), with a reversible solid/liquid change of state, having a melting point of greater than 30° C. which may be up to 200° C., a hardness of greater than 0.5 MPa and having an anisotropic crystalline organization in the solid state. It may be a hydrocarbon-based wax, a fluoro wax and/or a silicone wax and may be of animal, plant, mineral or synthetic origin. It may be chosen, for example, from beeswax, carnauba wax, candelilla wax, paraffin waxes, hydrogenated castor oil, silicone waxes or microcrystalline waxes, and mixtures thereof.

In particular, the wax may be present in the form of a wax-in-water emulsion.

The wax may be present in a composition according to the invention in a content ranging from 0.01% to 50% by weight, in particular from 0.1% to 30% by weight and especially from 0.2% to 20% by weight relative to the total weight of the composition.

Surfactants

The composition of foundation type according to the invention, especially the base composition and/or the surface composition of the product of foundation type according to the invention, may also contain emulsifying surfactants present especially in a proportion ranging from 0.1 to 30% by weight and better still from 5% to 15% by weight relative to the total weight of the composition.

These surfactants may be chosen from anionic and nonionic surfactants. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of this reference, for the anionic and nonionic surfactants.

The surfactants preferably used in the composition of foundation type according to the invention are chosen from:

nonionic surfactants: fatty acids, fatty alcohols, polyethoxylated or polyglycerolated fatty alcohols such as polyethoxylated stearyl or cetylstearyl alcohol, fatty acid esters of sucrose, alkylglucose esters, in particular polyoxyethylenated C₁-C₆ alkyl glucose fatty esters, and mixtures thereof,

anionic surfactants: C₁₆-C₃₀ fatty acids neutralized with amines, aqueous ammonia or alkaline salts, and mixtures thereof.

Surfactants that allow oil-in-water or wax-in-water emulsions to be obtained are preferably used.

Film-Forming Polymer

The composition of foundation type according to the invention, especially the base composition and/or the surface composition of the product of foundation type according to the invention, may also comprise at least one film-forming polymer.

In the present patent application, the term “film-forming polymer” means a polymer capable of forming, by itself or in the presence of an auxiliary film-forming agent, a continuous film that adheres to the skin.

A film-forming polymer capable of forming a hydrophobic film, i.e. a polymer whose film has a solubility in water at 25° C. of less than 1% by weight, is preferably used.

The film-forming polymer may especially be at least one polymer chosen from the group comprising:

water-soluble film-forming polymers,

aqueous dispersions of water-dispersible film-forming polymer particles, also known as “latices”; in this case, the composition of foundation type should comprise an aqueous phase,

liposoluble film-forming polymers,

lipodispersible film-forming polymers in the form of non-aqueous dispersions of polymer particles, preferably dispersions of polymer particles, where appropriate surface-stabilized with at least one stabilizer, in one or more silicone and/or hydrocarbon-based oils; these non-aqueous dispersions are also known as “NADs”.

The composition of foundation type may in parallel comprise a mixture of these polymers.

The film-forming polymer may be present in a composition according to the invention in a solids content ranging from 0.01% to 20% by weight and especially from 0.5% to 10% by weight relative to the total weight of the composition.

Among the film-forming polymers that may be used according to the invention, mention may be made of synthetic polymers, of free-radical type or of polycondensate type, polymers of natural origin, and mixtures thereof.

The term “free-radical film-forming polymer” means a polymer obtained by polymerization of unsaturated monomers, especially ethylenically unsaturated monomers, each monomer being capable of homopolymerizing (unlike polycondensates).

The film-forming polymers of free-radical type may especially be vinyl polymers or copolymers, especially acrylic polymers.

The vinyl film-forming polymers may result from the polymerization of ethylenically unsaturated monomers containing at least one acid group and/or esters of the acidic monomers and/or amides of these acidic monomers.

α,β-Ethylenic unsaturated carboxylic acids such as acrylic acid, methacrylic acid, crotonic acid, maleic acid or itaconic acid may be used as monomer bearing an acid group. (Meth)acrylic acid and crotonic acid are preferably used, and more preferably (meth)acrylic acid.

The esters of acidic monomers are advantageously chosen from (meth)acrylic acid esters (also known as (meth)acrylates), especially alkyl (meth)acrylates, in particular of a C₁-C₃₀ alkyl and preferably a C1-C20 alkyl, aryl (meth)acrylates, in particular of a C₆-C₁₀ aryl, and hydroxyalkyl (meth)acrylates, in particular of a C₂-C₆ hydroxyalkyl.

Among the alkyl (meth)acrylates that may be mentioned are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates that may be mentioned are hydroxyethyl acrylate, 2-hydroxypropyl acrylate, hydroxyethyl methacrylate and 2-hydroxypropyl methacrylate.

Among the aryl (meth)acrylates that may be mentioned are benzyl acrylate and phenyl acrylate.

The (meth)acrylic acid esters that are particularly preferred are alkyl (meth)acrylates.

According to the present invention, the alkyl group of the esters may be either fluorinated or perfluoriated, i.e. some or all of the hydrogen atoms of the alkyl group are replaced with fluorine atoms.

Examples of amides of the acidic monomers that may be mentioned include (meth)acrylamides, and especially N-alkyl(meth)acrylamides, in particular of a C2-C12 alkyl. Among the N-alkyl (meth)acrylamides that may be mentioned are N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. In particular, these monomers may be polymerized with acidic monomers and/or esters thereof and/or amides thereof, such as those mentioned previously.

Examples of vinyl esters that may be mentioned include vinyl acetate, vinyl neodecanoate, vinyl pivalate, vinyl benzoate and vinyl t-butylbenzoate.

Styrene monomers that may be mentioned include styrene and α-methylstyrene.

It is possible to use any monomer known to those skilled in the art falling within the categories of acrylic and vinyl monomers (including monomers modified with a silicone chain).

Among the film-forming polycondensates that may be mentioned are polyurethanes, polyesters, polyesteramides, polyamides, epoxyester resins and polyureas.

The polyurethanes may be chosen from anionic, cationic, nonionic and amphoteric polyurethanes, polyurethane-acrylics, polyurethanes-polyvinylpyrrolidones, polyester-polyurethanes, polyether-polyurethanes, polyureas and polyurea-polyurethanes, and mixtures thereof.

The polymers of natural origin, optionally modified, may be chosen from shellac resin, sandarac gum, dammar resins, elemi gums, copal resins and cellulose-based polymers, and mixtures thereof.

In a first embodiment of the composition of foundation type according to the invention, the film-forming polymer may be present in the form of particles in aqueous dispersion, which are generally known as latices or pseudolatices. The techniques for preparing these dispersions are well known to those skilled in the art.

Aqueous dispersions of film-forming polymers that may be used include the acrylic dispersions sold under the names Neocryl XK-90®, Neocryl A-1070®, Neocryl A-1090®, Neocryl BT-62®, Neocryl A-1079®, Neocryl A-523® by the company Avecia-Neoresins, Dow Latex 432® by the company Dow Chemical, Daitosol 5000 AD® by the company Daito Kasey Kogyo; or alternatively the aqueous polyurethane dispersions sold under the names Neorez R-981®, Neorez R-974® by the company Avecia-Neoresins, Avalure UR-405®, Avalure UR-410®, Avalure UR-425®, Avalure UR-450®, Sancure 875®, Sancure 861®, Sancure 878®, Sancure 2060® by the company Goodrich, Impranil 85® by the company Bayer and Aquamere H-1511® by the company Hydromer.

Aqueous dispersions of film-forming polymers that may also be used include the polymer dispersions resulting from the free-radical polymerization of one or more free-radical monomers within and/or partially at the surface of preexisting particles of at least one polymer chosen from the group consisting of polyurethanes, polyureas, polyesters, polyesteramides and/or alkyds. These polymers are generally known as hybrid polymers.

In a second embodiment of the composition of foundation type according to the invention, the film-forming polymer may be a water-soluble polymer and is thus present in the aqueous phase of the composition in dissolved form. Examples of water-soluble film-forming polymers that may be mentioned include:

proteins, for instance proteins of plant origin such as wheat or soybean proteins; proteins of animal origin such as keratins, for example keratin hydrolysates and sulphonic keratins;

anionic, cationic, amphoteric or nonionic chitin or chitosan polymers;

cellulose polymers such as hydroxyethylcellulose, hydroxypropylcellulose, methylcellulose, ethylhydroxyethylcellulose, carboxymethylcellulose and quaternized cellulose derivatives;

acrylic polymers or copolymers, such as polyacrylates or polymethacrylates;

vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate; copolymers of vinylpyrrolidone and of vinylcaprolactam; polyvinyl alcohol;

polymers of natural origin, optionally modified, such as:

gum arabic, guar gum, xanthan derivatives and karaya gum;

alginates and carrageenans;

glycosaminoglycans, and hyaluronic acid and its derivatives;

shellac resin, sandarac gum, dammar resins, elemi gums and copal resins;

deoxyribonucleic acid;

mucopolysaccharides such as hyaluronic acid, chondroitin sulphates, and mixtures thereof.

According to another embodiment variant of the composition of foundation type according to the invention, the film-forming polymer may be present in a liquid fatty phase comprising organic oils or solvents such as those described above. For the purposes of the invention, the term “liquid fatty phase” means a fatty phase that is liquid at room temperature (25° C.) and atmospheric pressure (760 mmHg, i.e. 105 Pa), composed of one or more fatty substances that are liquid at room temperature, also known as oils, which are generally mutually compatible.

Preferably, the liquid fatty phase comprises a volatile oil, optionally mixed with a non-volatile oil, the oils possibly being chosen from the oils mentioned above.

In a third embodiment of the composition of foundation type according to the invention, the film-forming polymer may be in the form of surface-stabilized particles dispersed in the liquid fatty phase.

The dispersion of surface-stabilized polymer particles may be manufactured as described in document EP-A-749 747.

The polymer particles are surface-stabilized by means of a stabilizer, which may be a block polymer, a grafted polymer and/or a random polymer, alone or as a mixture.

Dispersions of film-forming polymer in the liquid fatty phase, in the presence of stabilizers, are described especially in documents EP-A-749 746, EP-A-923 928 and EP-A-930 060, the content of which is incorporated by reference into the present patent application.

The size of the polymer particles in dispersion either in the aqueous phase or in the liquid fatty phase may range from 5 nm to 600 nm and preferably from 20nm to 300 nm.

In a fourth embodiment of the composition of foundation type according to the invention, the film-forming polymer may be dissolved in the liquid fatty phase, in which case the film-forming polymer is said to be a liposoluble polymer.

Examples of liposoluble polymers that may be mentioned include copolymers of a vinyl ester (the vinyl group being directly attached to the oxygen atom of the ester group and the vinyl ester having a saturated, linear or branched hydrocarbon-based radical of 1 to 19 carbon atoms, linked to the carbonyl of the ester group) and of at least one other monomer, which may be a vinyl ester (other than the vinyl ester already present), an α-olefin (containing from 8 to 28 carbon atoms), an alkyl vinyl ether (the alkyl group of which contains from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked using crosslinking agents, which have the aim [lacuna], which may be either of the vinyl type or of the allylic or methallylic type, such as tetraallyloxyethane, divinylbenzene, divinyl octanedioate, divinyl dodecanedioate, and divinyl octadecanedioate.

Examples of these copolymers that may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethyl-pentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene crosslinked with 0.2% divinylbenzene, and allyl propionate/allyl stearate, crosslinked with 0.2% divinylbenzene.

Liposoluble film-forming polymers that may also be mentioned include liposoluble homopolymers, and in particular those resulting from the homopolymerization of vinyl esters containing from 9 to 22 carbon atoms or of alkyl acrylates or methacrylates, the alkyl radicals containing from 10 to 20 carbon atoms.

Such liposoluble homopolymers may be chosen from polyvinyl stearate, polyvinyl stearate crosslinked with divinylbenzene, with diallyl ether or with diallyl phthalate, polystearyl (meth)acrylate, polyvinyl laurate and polylauryl (meth)acrylate, these poly(meth)acrylates possibly being crosslinked with ethylene glycol dimethacrylate or tetraethylene glycol dimethacrylate.

The liposoluble copolymers and homopolymers defined above are known and described especially in patent application FR-A-2 232 303; they may have a weight-average molecular weight ranging from 2000 to 500,000 and preferably from 4000 to 200,000.

As liposoluble film-forming polymers that may be used in the invention, mention may also be made of polyalkylenes and especially copolymers of C₂-C₂₀ alkenes, for instance polybutene, alkylcelluloses with a saturated or unsaturated, linear or branched C₁ to C₈ alkyl radical, for instance ethylcellulose and propylcellulose, vinylpyrrolidone (VP) copolymers and especially copolymers of vinylpyrrolidone and of a C₂ to C₄₀ and better still C₃ to C₂₀ alkene. As examples of VP copolymers that may be used in the invention, mention may be made of the VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer.

The composition according to the invention may also comprise an auxiliary film-forming agent that promotes the formation of a film with the film-forming polymer. Such a film-forming agent may be chosen from any compound known to the those skilled in the art as being capable of satisfying the desired function, and may be chosen especially from plasticizers and coalescers.

The composition or product of foundation type of the invention is generally in the form of a foundation to be applied especially to the face or the neck, a concealer product, a complexion corrector, a tinted cream or makeup base for the face or a makeup composition for the body.

The composition of foundation type of the invention, especially the base composition and/or the surface composition of the product of foundation type according to the invention, may be in a solid form, for example in pulverulent, compacted or cast form or in the form of a stick or in the form of a fluid, for example a pasty or liquid fluid. It may also be in the form of a soft paste, an ointment or a solid or fluid pomade of cream type. For example, it may be an oil-in-water or water-in-oil emulsion, a gel, especially a solid or soft anhydrous gel, and may even be in two-phase form. According to this variant, it is more particularly in the form of a foundation with an oily and especially anhydrous continuous phase; in this case, it may contain an aqueous phase in a content of less than 5%.

In the case of the product according to the invention, the two corresponding compositions may be in identical or different forms, and especially in accordance with the foregoing text.

The composition of foundation type according to the invention, especially the base composition and/or the surface composition of the product of foundation type according to the invention, may be manufactured by the known processes generally used in cosmetics.

According to another of its aspects, the invention also relates to the use of a composition or a product as defined above, to lighten a dark skin.

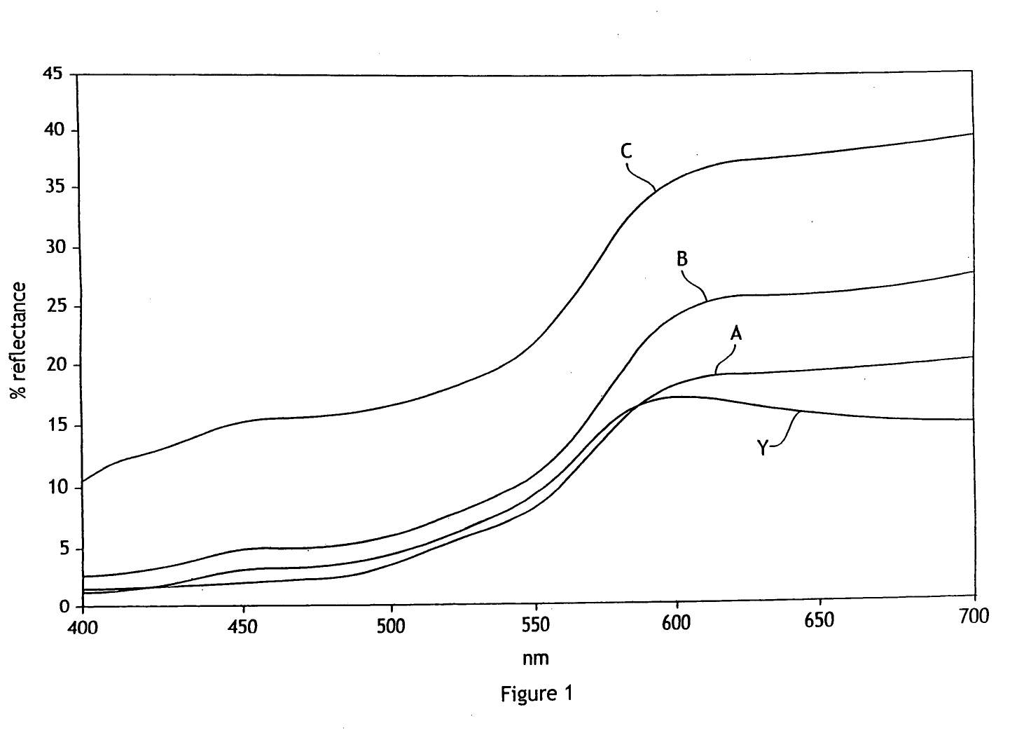

The invention may be understood more clearly on reading the detailed description that follows, of non-limiting examples of implementation of this invention, and FIG. 1:

FIG. 1: representation of the reflectance spectra of compositions A, B, C and Y.

### EXAMPLES

12 foundation compositions were prepared as follows, the proportions therein being expressed as percentages by mass:

Composition A

This is a foundation that is more particularly intended for dark skins. Its formulation is as follows:

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - | 4.48% |
| viscosity: 500 CST)(KF-6017 from the company | |
| Shin Etsu Silicones) | |
| isoeicosane (Permethyl 102 A from Permethyl) | 2% |
| polydimethylsiloxane (viscosity: 5 CST) | 2% |
| (Fluid 200 5 CS from the company Dow Corning) | |
| isostearyl neopentanoate | 0.50% |
| cyclohexadimethylsiloxane | 8% |
| cyclopentadimethylsiloxane | 11.36% |
| isododecane | 13% |
| D,L-α-tocopherol (vitamin E) | 0.08% |
| hectorite modified with distearyldimethylammonium | 1.60% |
| chloride (Bentone 38V from the company Elementis) | |
| yellow iron oxide coated with perfluoroalkyl | 1.86% |
| phosphate, as a dispersion at 50% by weight in | |
| decamethylcyclopentasiloxane/dimethicone copolyol | |
| (CI: 77492) (FA50DYF from the company Kobo) | |
| brown iron oxide coated with perfluoroalkyl | 0.72% |
| phosphate, as a dispersion at 50% by weight in | |
| cyclomethicone/dimethylpolysiloxane copolyol | |
| (CI: 77491) (FA50DRF from the company Kobo) | |
| black iron oxide coated with perfluoroalkyl | 0.34% |
| phosphate, as a dispersion at 65% by weight in | |
| cyclomethicone/dimethylpolysiloxane copolyol | |
| (CI: 77499) (FA65DBF from the company Kobo) | |
| alumina-treated titanium oxide coated with | 7.39% |
| perfluoroalkyl phosphate, as a | |
| dispersion at 65% by weight in | |
| decamethylcyclopentasiloxane/dimethiconecopolyol | |
| (CI 77891) (FA65DF from the company Kobo) | |
| hollow polymethyl methacrylate microspheres | 4% |
| (particle size: 10 to 12 microns), (Covabead LH 85 | |
| from the company Wackherr) | |
| 1,3-butylene glycol | 10% |
| sodium chloride | 0.70% |
| preserving agents | 0.90% |
| mica-brown iron oxide (60/40) (CI: 77019 + 77491) | 2% |
| (Colourona Passion Orange from the company Merck) | |
| disodium salt of tartrazine (CI: 19140) | 2% |
| (FD & C Yellow 5 from the company LCW) | |
| water | quantity |
| | sufficient |
| | for (qs) |
| | 100% |

Composition B

This is a foundation that is more particularly suitable for light dark skins. Its formulation is as follows:

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST)(KF-6017 from the company Shin Etsu Silicones) | |
| isoeicosane (Permethyl 102 A from Permethyl) | 2% |
| polydimethylsiloxane (viscosity: 5 CST) | 2% |
| (Fluid 200 5 CS from the company Dow Corning) | |
| isostearyl neopentanoate | 0.50% |
| cyclohexadimethylsiloxane | 8% |
| cyclopentadimethylsiloxane | 11.36% |
| isododecane | 13% |
| D,L-α-tocopherol (vitamin E) | 0.08% |
| hectorite modified with distearyldimethylammonium | 1.60% |
| chloride (Bentone 38V from the company Elementis) | |
| yellow iron oxide coated with perfluoroalkyl phosphate, | 6.90% |
| as a dispersion at 50% by weight in | |
| decamethylcyclopentasiloxane/dimethicone | |
| copolyol (CI: 77492) (FA50DYF from the company Kobo) | |
| mica-bismuth oxychloride-brown iron oxide (47/28/25) | 6.81% |
| (Chroma - Lite Brown from the company Engelhard) | |
| aluminium lake of red Allura on alumina (40/60) | 0.60% |
| (FD&C Red 40 A1 lake from the company Noveon) | |
| hollow polymethyl methacrylate microspheres | 4% |
| (particle size: 10 to 12 microns), (Covabead LH 85 | |
| from the company Wackherr) | |
| 1,3-butylene glycol | 10% |
| sodium chloride | 0.70% |
| preserving agents | 0.90% |
| water | qs 100% |

Composition C

This composition is most particularly suitable for black skins. Its formulation is as follows:

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST) (KF-6017 from the company Shin Etsu Silicones) | |
| isoeicosane (Permethyl 102 A) | 2% |
| polydimethylsiloxane (viscosity: 5 CST) | 2% |
| (Fluid 200 5 CS from the company Dow Corning) | |
| isostearyl neopentanoate | 0.50% |
| cyclohexadimethylsiloxane | 8% |
| cyclopentadimethylsiloxane | 11.36% |
| isododecane | 13% |
| D,L-α-tocopherol (vitamin E) | 0.08% |
| hectorite modified with distearyldimethylammonium | 1.60% |
| chloride (Bentone 38V from the company Elementis) | |
| yellow iron oxide coated with perfluoroalkyl phosphate, | 1.86% |
| as a dispersion at 50% by weight in | |
| decamethylcyclopentasiloxane/dimethicone | |
| copolyol (CI: 77492) (FA50DYF from the company Kobo) | |
| brown iron oxide coated with perfluoroalkyl phosphate, | 0.72% |
| as a dispersion at 50% by weight in | |
| cyclomethicone/dimethylpolysiloxane | |
| copolyol (CI: 77491) (FA50DRF from the company Kobo) | |
| black iron oxide coated with perfluoroalkyl phosphate, | 0.34% |
| as a dispersion at 65% by weight in | |
| cyclomethicone/dimethylpolysiloxane | |
| copolyol (CI: 77499) (FA65DBF from the company Kobo) | |
| alumina-treated titanium oxide coated with perfluoroalkyl | 7.39% |
| phosphate, as a dispersion at 65% by weight in | |
| decamethylcyclopentasiloxane/dimethiconecopolyol | |
| (CI 77891) (FA65DF from the company Kobo) | |
| hollow polymethyl methacrylate microspheres | 4% |
| (particle size: 10 to 12 microns), (Covabead LH 85 | |
| from the company Wackherr) | |
| 1,3-butylene glycol | 10% |
| sodium chloride | 0.70% |
| preserving agents | 0.90% |
| silica-brown iron oxide (Xirona Indian Summer from Merck) | 4% |
| water | qs 100% |

Composition D

This is a foundation composition that is more particularly suitable for black skins. Its formulation is as follows:

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST)(KF-6017 from the company Shin Etsu Silicones) | |
| isoeicosane (Permethyl 102 A) | 2% |
| polydimethylsiloxane (viscosity: 5 CST) | 2% |
| (Fluid 200 5 CS from the company Dow Corning) | |
| isostearyl neopentanoate | 0.50% |
| cyclohexadimethylsiloxane | 8% |
| cyclopentadimethylsiloxane | 11.36% |
| isododecane | 13% |
| D,L-α-tocopherol (vitamin E) | 0.08% |
| hectorite modified with distearyldimethylammonium chloride | 1.60% |
| (Bentone 38V from the company Elementis) | |
| yellow iron oxide coated with perfluoroalkyl phosphate, | 5.25% |
| as a dispersion at 50% by weight in | |
| decamethylcyclopentasiloxane/dimethicone | |
| copolyol (CI: 77492) (FA50DYF from the company Kobo) | |
| brown iron oxide coated with perfluoroalkyl phosphate, | 0.72% |
| as a dispersion at 50% by weight in | |
| cyclomethicone/dimethylpolysiloxane | |
| copolyol (CI: 77491) (FA50DRF from the company Kobo) | |
| black iron oxide coated with perfluoroalkyl phosphate, | 0.34% |
| as a dispersion at 65% by weight in | |
| cyclomethicone/dimethylpolysiloxane | |
| copolyol (CI: 77491) (FA65DBF from the company Kobo) | |
| alumina-treated titanium oxide coated with perfluoroalkyl | 4% |
| phosphate, as a dispersion at 65% by weight in | |
| decamethylcyclopentasiloxane/dimethiconecopolyol | |
| (CI 77891) (FA65DF from the company Kobo) | |
| hollow polymethyl methacrylate microspheres | 4% |
| (particle size: 10 to 12 microns), (Covabead LH 85 | |
| from the company Wackherr) | |
| 1,3-butylene glycol | 10% |
| sodium chloride | 0.70% |
| silica-brown iron oxide (Xirona Indian Summer from Merck) | 4% |
| preserving agents | 0.90% |
| water | qs 100% |

Composition E

This is a stick for black skin. Its formulation is as follows:

| | |
|---|---|
| polyethylene wax (MW: 500) (Polywax 500 from Bareco) | 4% |
| ethylene homopolymer (melting point: 79.5° C.) | 8% |
| (Performalene 400 from New Phase Technologies) | |
| cyclopentadimethylsiloxane | 5% |
| cyclohexadimethylsiloxane | 20% |
| isododecane | 19% |
| phenyl trimethylsiloxy trisiloxane (viscosity: 20 CST - | 19% |
| PM: 372)(DC556 from Dow Corning) | |
| hollow polymethyl methacrylate microspheres (particle | 10% |
| size: 10 to 12 microns) (Covabead LH85 from Wackherr) | |
| mica-brown iron oxide (94/6) (CI: 77019 + 77491) | 12% |
| (Cosmetica Orange from Engelhard) | |
| yellow iron oxide coated with perfluoroalkyl phosphate, | 3% |
| as a dispersion at 50% by weight in | |
| decamethylcyclopentasiloxane/dimethicone | |
| copolyol (CI: 77492) (FA50DYF from the company Kobo) | |

Composition F

This is a fluid foundation for black skins. Its formulation is as follows:

| | |
|---|---|
| cetyl polyethylene glycol/PPG - 10/1-dimethicone | 0.80% |
| (Abil EM 90) | |
| polyglyceryl 4-isostearate (Isolan GI34 from the | 0.60% |
| company Goldschmidt) | |
| hexyl laurate | 0.60% |
| PEG-10 dimethicone (KF6017) | 4.48% |
| isoeicosane (Permethyl 102A) | 2% |
| dimethicone (DC 200 Fluid) | 2% |
| isostearyl neopentanoate | 0.50% |
| cyclohexasiloxane | 8% |
| cyclopentasiloxane | 11.36% |
| isododecane | 13% |
| D, L-α-tocopherol (vitamin E) | 0.08% |
| hectorite modified with distearyldimethylammonium | 1.6% |
| chloride (Bentone 38V from the company Elementis) | |
| iron oxides and cyclopentasiloxane and PEG/PPG-18/ | 4.73% |
| 18-dimethicone and C₉ to C₁₅ fluoroalkyl phosphates | |
| (FA5ODYF from Kobo) | |
| iron oxides and cyclopentasiloxane and PEG/PPG-18/ | 2.595% |
| 18-dimethicone and C₉ to C₁₅ fluoroalkyl phosphates | |
| (FA50DRF from Kobo) | |
| iron oxides and cyclopentasiloxane and PEG/PPG-18/ | 1.22% |
| 18-dimethicone and C₉ to C₁₅ fluoroalkyl phosphates | |
| (FA65DBF from Kobo) | |
| titanium dioxide and cyclopentasiloxane and PEG/ | 1.765% |
| PPG-18/18-dimethicone and C₉ to C₁₅ fluoroalkyl | |
| phosphates and alumina (FA65DF from Kobo) | |
| polymethyl methacrylate (Covabead LH 85) | 4% |
| butylene glycol | 10% |
| sodium chloride | 0.70% |
| preserving agents | 0.90% |
| mica and iron oxides (Colourona Passion Orange | 2% |
| from Merck) | |
| Yellow-6 lake (pigments: FDC Yellow 6 A1 Lake | 2% |
| from Sun Chemical) (CI15985) | |
| water | qs 100% |

Composition Y

This is a foundation that is more particularly intended for dark skins. Its formulation is as follows:

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST)(KF-6017 from the company Shin Etsu Silicones) | |
| isoeicosane (Permethyl 102 A from Permethyl) | 2% |
| polydimethylsiloxane (viscosity: 5 CST) | 2% |
| (Fluid 200 5 CS from the company Dow Corning) | |
| isostearyl neopentanoate | 0.5% |
| cyclohexadimethylsiloxane | 8% |
| cyclopentadimethylsiloxane | 11.3% |
| isododecane | 13% |
| D,L-α-tocopherol (vitamin E) | 0.08% |
| hectorite modified with distearyldimethylammonium chloride | 1.60% |
| (Bentone 38V from the company Elementis) | |
| yellow iron oxide coated with perfluoroalkyl phosphate, | 10.93% |
| as a dispersion at 50% by weight in | |
| decamethylcyclopentasiloxane/dimethicone copolyol | |
| (CI: 77492) (FA50DYF from the company Kobo) | |
| brown iron oxide coated with perfluoroalkyl phosphate, | 2.15% |
| as a dispersion at 50% by weight in | |
| cyclomethicone/dimethylpolysiloxane copolyol | |
| (CI: 77491) (FA50DRF from the company Kobo) | |
| hollow polymethyl methacrylate microspheres (particle | 4% |
| size: 10 to 12 microns) (Covabead LH 85 from Wackherr) | |
| mica-ferric blue sold under the name | 1.23% |
| Microna Matte Blue by the company Merck | |
| 1,3-butylene glycol | 10% |
| sodium chloride | 0.70% |
| preserving agents | 0.90% |
| water | qs 100% |

Composition G

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST)(KF-6017 from the company Shin Etsu Silicones) | |
| isoeicosane (Permethyl 102 A from Permethyl) | 2% |
| polydimethylsiloxane (viscosity: 5 CST) | 2% |
| (Fluid 200 5 CS from the company Dow Corning) | |
| isostearyl neopentanoate | 0.5% |
| cyclohexadimethylsiloxane | 8% |
| isododecane | 13% |
| D,L-α-tocopherol (vitamin E) | 0.08% |
| Smectite: modified magnesium silicate in | 12.96% |
| cyclopentadimethylsiloxane and ethanol (Bentone VS 5 V gel) | |
| yellow iron oxide coated with perfluoroalkyl phosphate | 1.28% |
| (PF 5 Yellow 601 from Daito) | |
| brown iron oxide coated with perfluoroalkyl phosphate | 2.13% |
| (PF 5 Red R 516 L from Daito) | |
| black iron oxide coated with perfluoroalkyl phosphate | 0.47% |
| (PF 5 Black BL 100 from Daito) | |
| ultramarine blue coated with perfluoroalkyl phosphate | 1.28% |
| (PF 5 Ultramarine No. 801 from Daito) | |
| cyclopentadimethylsiloxane (Dow Corning 245 Fluid | 5.17% |
| from Dow Corning) | |
| hollow polymethyl methacrylate microspheres (particle size: | 4% |
| 10 to 12 μm), (Covabead LH85 from the company Wackherr) | |
| 1,3-butylene glycol | 10% |
| sodium chloride | 0.7% |
| preserving agents | 0.90% |
| phenoxyethanol (Phenoxetol) | 0.5% |
| mica-brown iron oxide (60/40) (CI: 77019 + 77491) | 2% |
| (Colourona Passion Orange from the company Merck) | |
| Yellow-6 lake (pigments : FDC Yellow 6 A1 Lake from | 2% |
| Sun Chemicals) (CI15985) | |
| water | qs 100% |

Composition H

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST)(KF-6017 from the company Shin Etsu Silicones) | |
| isoeicosane (Permethyl 102 A from Permethyl) | 2% |
| polydimethylsiloxane (viscosity: 5 CST) | 2% |
| (Fluid 200 5 CS from the company Dow Corning) | |
| isostearyl neopentanoate | 0.5% |
| cyclohexadimethylsiloxane | 8% |
| isododecane | 13% |
| D,L-α-tocopherol (vitamin E) | 0.08% |
| Smectite: modified magnesium silicate in | 12.96% |
| cyclopentadimethylsiloxane and ethanol (Bentone VS 5 V gel) | |
| yellow iron oxide coated with perfluoroalkyl phosphate | 2.37% |
| (PF 5 Yellow 601 from Daito) | |
| brown iron oxide coated with perfluoroalkyl phosphate | 1.3% |
| (PF 5 Red R 516 L from Daito) | |
| black iron oxide coated with perfluoroalkyl phosphate | 0.61% |
| (PF 5 Black BL 100 from Daito) | |
| titanium oxide coated with perfluoroalkyl phosphate | 0.88% |
| (PF 5 Ultramarine No. 801 from Daito) | |
| cyclopentadimethylsiloxane (Dow Corning 245 Fluid | 5.13% |
| from Dow Corning) | |
| hollow polymethyl methacrylate microspheres (particle size: | 4% |
| 10 to 12 μm), (Covabead LH85 from the company Wackherr) | |
| 1,3-butylene glycol | 10% |
| sodium chloride | 0.7% |
| preserving agents | 0.90% |
| phenoxyethanol (Phenoxetol) | 0.5% |
| mica-brown iron oxide (60/40) (CI: 77019 + 77491) | 2% |
| (Colourona Passion Orange from the company Merck) | |
| Yellow-6 lake (pigments: FDC Yellow 6 A1 Lake from | 2% |
| Sun Chemicals) (CI15985) | |
| water | qs 100% |

Composition I

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - viscosity: | 4.48% |
| 500 CST)(KF-6017 from the company Shin Etsu Silicones) | |
| isoeicosane (Permethyl 102 A from Permethyl) | 2% |
| polydimethylsiloxane (viscosity: 5 CST) | 2% |
| (Fluid 200 5 CS from the company Dow Coming) | |
| isostearyl neopentanoate | 0.5% |
| cyclohexadimethylsiloxane | 8% |
| isododecane | 13% |
| D,L-α-tocopherol (vitamin E) | 0.08% |
| Smectite: modified magnesium silicate in | 12.96% |
| cyclopentadimethylsiloxane and ethanol (Bentone VS 5 V gel) | |
| yellow iron oxide coated with perfluoroalkyl phosphate | 3.46% |
| (PF 5 Yellow 601 from Daito) | |
| cyclopentadimethylsiloxane (Dow Corning 245 Fluid | 3.46% |
| from Dow Corning) | |
| hollow polymethyl methacrylate microspheres (particle size: | 4% |
| 10 to 12 μm), (Covabead LH85 from the company Wackherr) | |
| 1,3-butylene glycol | 10% |
| sodium chloride | 0.7% |
| preserving agents | 0.90% |
| phenoxyethanol (Phenoxetol) | 0.5% |
| mica-bismuth oxychloride brown iron oxide | 6.81% |
| (Chroma-Lite Brown 4509 from Engelhard) | |
| aluminium lake of red allura on alumina | 0.6% |
| (FDC Red 40 AL Lake - 6808 from Noveon) | |
| water | qs 100% |

Composition J

| | |
|---|---|
| oxyethylenated polymethylcetyldimethylmethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - | 4.48% |
| viscosity: 500 CST)(KF-6017 from the company | |
| Shin Etsu Silicones) | |
| isoeicosane (Permethyl 102 A from Permethyl) | 2% |
| polydimethylsiloxane (viscosity : 5 CST) | 2% |
| (Fluid 200 5 CS from the company Dow Corning) | |
| isostearyl neopentanoate | 0.5% |
| cyclohexadimethylsiloxane | 8% |
| isododecane | 13% |
| D,L-α-tocopherol (vitamin E) | 0.08% |
| Smectite: modified magnesium silicate in | 12.96% |
| cyclopentadimethylsiloxane and ethanol | |
| (Bentone VS 5 V gel) | |
| yellow iron oxide coated with perfluoroalkyl phosphate | 1.75% |
| (PF 5 Yellow 601 from Daito) | |
| brown iron oxide coated with perfluoroalkyl phosphate | 0.39% |
| (PF 5 Red R 516 L from Daito) | |
| black iron oxide coated with perfluoroalkyl phosphate | 0.14% |
| (PF 5 Black BL 100 from Daito) | |
| titanium oxide coated with perfluoroalkyl phosphate | 2.22% |
| (PF 5 TiO2 A 100 from Daito) | |
| cyclopentadimethylsiloxane (Dow Corning 245 Fluid | 4.49% |
| from Dow Corning) | |
| hollow polymethyl methacrylate microspheres | 4% |
| (particle size: 10 to 12 μm), | |
| (Covabead LH85 from the company Wackherr) | |
| 1,3-butylene glycol | 10% |
| sodium chloride | 0.7% |
| preserving agents | 0.90% |
| phenoxyethanol (Phenoxetol) | 0.5% |
| silica-brown iron oxide (Xirona Indian Summer from Merck) | 3% |
| mica-bismuth oxychloride-yellow iron oxide (Chroma-Lite | 2.32% |
| Yellow 4502 from Engelhard) | |
| water | qs 100% |

Composition K

| | |
|---|---|
| oxyethylenated polymethylcetyldimethylsiloxane | 0.80% |
| (Abil EM 90 from the company Goldschmidt) | |
| polyglycerol isostearate (4 mol) | 0.60% |
| (Isolan GI 34 from the company Goldschmidt) | |
| hexyl laurate | 0.60% |
| oxyethylenated polydimethylsiloxane (DP: 70 - | 4.48% |
| viscosity: 500 CST)(KF-6017 from the company | |
| Shin Etsu Silicones) | |
| isoeicosane (Permethyl 102 A from Permethyl) | 2% |
| polydimethylsiloxane (viscosity: 5 CST) | 2% |
| (Fluid 200 5 CS from the company Dow Corning) | |
| isostearyl neopentanoate | 0.5% |
| cyclohexadimethylsiloxane | 8% |
| isododecane | 13% |
| D,L-α-tocopherol (vitamin E) | 0.08% |
| Smectite: modified magnesium silicate in | 12.96% |
| cyclopentadimethylsiloxane and ethanol | |
| (Bentone VS 5 V gel) | |
| yellow iron oxide coated with perfluoroalkyl phosphate | 1.75% |
| (PF 5 Yellow 601 from Daito) | |
| brown iron oxide coated with perfluoroalkyl phosphate | 0.39% |
| (PF 5 Red R 516 L from Daito) | |
| black iron oxide coated with perfluoroalkyl phosphate | 0.19% |
| (PF 5 Black BL 100 from Daito) | |
| titanium oxide coated with perfluoroalkyl phosphate | 2.17% |
| (PF 5 TiO2 A 100 from Daito) | |
| cyclopentadimethylsiloxane (Dow Corning 245 Fluid | 4.49% |
| from Dow Corning) | |
| hollow polymethyl methacrylate microspheres | 4% |
| (particle size: 10 to 12 μm), | |
| (Covabead LH85 from the company Wackherr) | |
| 1,3-butylene glycol | 10% |
| sodium chloride | 0.7% |
| preserving agents | 0.90% |
| phenoxyethanol (Phenoxetol) | 0.5% |
| silica-brown iron oxide (Xirona Indian Summer from Merck) | 3.5% |
| mica-bismuth oxychloride-yellow iron oxide | 1.83% |
| (Chroma-Lite Yellow 4502 from Engelhard) | |
| water | qs 100% |

### REFLECTANCE TEST

Dark skins were made up with compositions A, B, C and Y.

The color of each composition A, B, C, Y, G, H, I, J and K is given in the table below, the values being indicated with a precision of 15%, better still 10% and even better still 5%:

| Composition | L* | a* | b* | C* | h(°) |
|---|---|---|---|---|---|
| A | 39.46 | 18.63 | 37.11 | 41.52 | 63.34 |
| B | 48.59 | 20.21 | 31.91 | 37.77 | 57.65 |
| C | 57.24 | 14.69 | 19.65 | 24.53 | 53.22 |
| Y | 39.22 | 13.80 | 32.25 | 35.08 | 66.83 |
| G | 38.04 | 17.12 | 15.45 | 23.06 | 42.08 |
| H | 43.50 | 20.25 | 21.19 | 29.31 | 46.30 |
| I | 48.38 | 18.73 | 23.51 | 30.06 | 51.46 |
| J | 55.89 | 17.55 | 23.30 | 29.17 | 53 |
| K | 55.78 | 16.85 | 21.51 | 27.32 | 51.93 |

To measure the color of a composition, a color measurement in the bulk of the product was performed, as follows.

A cuvette referenced H247 was filled with the composition.

The contents were levelled off and a glass slide was then placed on the composition, taking care to avoid air bubbles under the slide. A color measurement was taken using a Minolta® spectrocolourimeter of CM-2002 type in medium aperture and in reflection mode, specular included.

A spectral reflectance measurement was also taken, in the bulk, using a Minolta® spectrocolourimeter of CM3700d type in reflection mode, specular excluded, UV included and with a small aperture d/8.

FIG. 1 shows the reflectance spectra for compositions A, B, C and Y present in the same dishes as above.

It is noted that, for the wavelength region between 600 and 680 nm, the reflectance is between 10% and 45% and more specifically between 12% and 40% for the four test compositions.

The reflectance is less than 20% in the range from 450 to 500 nm.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A composition of foundation type comprising, in a physiologically acceptable medium, at least one coloring agent and reflective particles, said composition having a hue angle h ranging from 40° to 70°, and a saturation C* ranging from 20 to 50.

2. The composition according to claim 1, said composition having a hue angle ranging from 50° to 70°.

3. The composition according to claim 1, comprising from 0.5% to 30% by weight of the at least one coloring agent, relative to the total weight of the composition.

4. The composition according to claim 1, comprising from 2 to 20% by weight of the at least one coloring agent, relative to the total weight of the composition.

5. The composition according to claim 1, comprising from 5 to 18% by weight of the at least one coloring agent, relative to the total weight of the composition.

6. The composition according to claim 1, wherein the coloring agent is the sole coloring agent and produces a yellow or orange colored effect.

7. The composition according to claim 1, said composition containing several coloring agents and their mixture produces a yellow or orange colored effect.

8. The composition according to claim 1, comprising from 0.5% to 60% by weight of reflective particles, relative to the total weight of the composition.

9. The composition according to claim 1, comprising from 1 to 30% by weight of reflective particles, relative to the total weight of the composition.

10. The composition according to claim 1, comprising from 2 to 20% by weight of reflective particles, relative to the total weight of the composition.

11. The composition according to claim 1, comprising from 3 to 10% by weight of reflective particles, relative to the total weight of the composition.

12. The composition according to claim 1, wherein the reflective particles have a color or glint selected from the group consisting of yellow, pink, red, bronze, orange, brown, gold and coppery.

13. The composition according to claim 1, wherein the said coloring agent is selected from the group consisting of mineral or organic pigments, coloring polymers, water-soluble or liposoluble dyes, organic lakes, metallic powders, and mixtures thereof.

14. The composition according to claim 13, wherein the mineral pigments are selected from the group consisting of yellow, red, and brown metal oxides.

15. The composition according to claim 13, wherein the mineral pigments are chosen among iron oxides.

16. The composition according to claim 13, wherein the water-soluble dye is selected from the group consisting of: brown dye identified by the name “caramel” according to the Color Index; yellow dyes identified by the Color Index numbers 10316, 13015, 18690, 18820, 18965, 19140, 45430, 47005, 75100 and known as Lactoflavin; the orange dyes identified by Color Index numbers 14270, 15510, 15980, 15985, 16230, 20170, 40215; red dyes identified by the Color Index numbers 14700, 14720, 14815, 15620, 16035, 16185, 16255, 16290, 17200, 18050, 18130, 18736, 24790, 27290, 45100, 45220, 45380, 45405, 45410, 45425, 45430, 75470; and mixtures thereof.

17. The composition according to claim 13, wherein the liposoluble dye is selected from the group consisting of: brown dye identified by Color Index number 12010; yellow dyes identified by Color Index numbers 12700, 21230, 47000, 75125, 75135; orange dyes identified by Color Index numbers 11920, 40800, 40820, 40825, 40850, 45396, 75120, 75130 and known as capasanthine; red dye identified by Color Index number 12150; and mixtures thereof.

18. The composition according to claim 1, wherein the at least one coloring agent is surface-treated.

19. The composition according to claim 1, wherein the reflective particles comprise particles comprising a natural or synthetic substrate at least partially coated with at least one layer of at least one metal or metallic compound.

20. The composition according to claim 19, wherein the substrate is selected from the group consisting of mono-material substrates, multi-material substrates, organic substrates and mineral substrates.

21. The composition according to claim 20, wherein the mineral substrate is selected from the group consisting of glasses, ceramics, graphite, metal oxides, aluminas, silicas, silicates, synthetic mica and mixtures thereof.

22. The composition according to claim 19, wherein the metallic compound is selected from the group consisting of titanium oxides, iron oxides, tin oxide, chromium oxide, barium sulphate, MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂ and mixtures or alloys thereof.

23. The composition according to claim 1, wherein the reflective particles comprise at least one nacre.

24. The composition according to claim 23, wherein the nacre is selected from the group consisting of nacres of golden color, bronze nacres, orange nacres, brown nacres, nacres with a coppery glint, nacres with a red glint, nacres with a yellow glint, nacres of red hue with a golden glint, pink nacres, black nacres with a golden glint, blue nacres, golden-green nacres, pink nacres, orange nacres, and mixtures thereof.

25. The composition according to claim 1, wherein the reflective particles comprise at least one goniochromatic coloring agent.

26. The composition according to claim 1, comprising at least one aqueous phase.

27. The composition according to claim 1, comprising at least one fatty phase.

28. The composition according to claim 27, said composition being anhydrous.

29. The composition according to claim 27, wherein said fatty phase contains at least one of: (a) at least one fatty substance that is liquid at room temperature and at atmospheric pressure and (b) at least one fatty substance that is solid at room temperature and at atmospheric pressure.

30. The composition according to claim 29, wherein said fatty substance that is liquid at room temperature and at atmospheric pressure comprises at least one volatile or non-volatile oil or a mixture thereof.

31. The composition according to claim 29, wherein said fatty substance that is solid at room temperature and atmospheric pressure is elected from the group consisting of waxes, pasty fatty substances, gums, and mixtures thereof.

32. The composition according to claim 1, comprising at least one additional filler.

33. The composition according to claim 1, further comprising at least one film-forming polymer.

34. The composition according to claim 1, said composition being in a fluid form selected from the group consisting of: liquid, paste, direct or inverse emulsion and gel.

35. The composition according to claim 1, said composition being in a solid form selected from the group consisting of: compact, pulverulent, cast form, and in the form of a stick.

36. The composition according to the claim 35, said composition being in a form selected from the group consisting of: a foundation to be applied to the face or the neck, a concealer product, a complexion corrector, a tinted cream, a makeup base for the face and a makeup composition for the body.

37. A composition of foundation type comprising, in a physiologically acceptable medium, at least one coloring agent and reflective particles, said composition having a reflectance ranging from 10% to 45% in the range of from 600 to 680 nm.

38. The composition according to claim 37, wherein the reflectance ranges from 12% to 40% in the range from 600 to 680 nm.

39. The composition according to claim 37, wherein the reflectance is less than 20% in the range from 450 to 500 nm.

40. The composition according to claim 37, comprising from 0.5% to 30% by weight of the at least one coloring agent, relative to the total weight of the composition.

41. The composition according to claim 37, comprising from 2 to 20% by weight of the at least one coloring agent, relative to the total weight of the composition.

42. The composition according to claim 37, comprising 5 to 18% by weight of the at least one coloring agent, relative to the total weight of the composition.

43. The composition according to claim 37, wherein the coloring agent is the sole coloring agent and produces a yellow or orange colored effect.

44. The composition according to claim 37, said composition containing several coloring agents and their mixture produces a yellow or orange colored effect.

45. The composition according to claim 37, comprising from 0.5% to 60% by weight of reflective particles, relative to the total weight of the composition.

46. The composition according to claim 37, comprising from 1 to 30% by weight of reflective particles, relative to the total weight of the composition.

47. The composition according to claim 37, comprising from 2 to 20% by weight of reflective particles, relative to the total weight of the composition.

48. The composition according to claim 37, comprising from 3 to 10% by weight of reflective particles, relative to the total weight of the composition.

49. The composition according to claim 37, wherein the reflective particles have a color or glint selected from the group consisting of yellow, pink, red, bronze, orange, brown, gold and coppery.

50. The composition according to claim 37, wherein said coloring agent is selected from the group consisting of mineral or organic pigments, coloring polymers, water-soluble or liposoluble dyes, organic lakes, metallic powders, and mixtures thereof.

51. The composition according to claim 50, wherein the mineral pigments are selected from the group consisting of yellow, red, and brown metal oxides.

52. The composition according to claim 50, wherein the mineral pigments are chosen among iron oxides.

53. The composition according to claim 50, wherein the water-soluble dye is selected from the group consisting of: brown dye identified by the name “caramel” according to the Color Index; yellow dyes identified by Color Index numbers 10316, 13015, 18690, 18820, 18965, 19140, 45430, 47005, 75100 and known as Lactoflavin; orange dyes identified by Color Index numbers 14270, 15510, 15980, 15985, 16230, 20170, 40215; red dyes identified by Color Index numbers 14700, 14720, 14815, 15620, 16035, 16185, 16255, 16290, 17200, 18050, 18130, 18736, 24790, 27290, 45100, 45220, 45380, 45405, 45410, 45425, 45430, 75470, 75470 and mixtures thereof.

54. The composition according to claim 50, wherein the liposoluble dye is selected from the group consisting of: brown dye identified by Color Index number 12010; yellow dyes identified, respectively, identified by Color Index numbers 12700, 21230, 47000, 75125, 75135; orange dyes identified by Color Index numbers 11920, 40800, 40820, 40825, 40850, 45396, 75120, 75130 and known as capasanthine; red dye identified by Color Index number 12150; and mixtures thereof.

55. The composition according to claim 37, wherein the at least one coloring agent is surface-treated.

56. The composition according to claim 37, wherein the reflective particles comprise particles comprising a natural or synthetic substrate at least partially coated with at least one layer of at least one metal or metallic compound.

57. The composition according to claim 56, wherein the substrate is selected from the group consisting of mono-material substrates, multi-material substrates, organic substrates and mineral substrates.

58. The composition according to claim 57, wherein the mineral substrate is selected from the group consisting of glasses, ceramics, graphite, metal oxides, aluminas, silicas, silicates, synthetic mica and mixtures thereof.

59. The composition according to claim 56, wherein the metallic compound is selected from the group consisting of titanium oxides, iron oxides, tin oxide, chromium oxide, barium sulphate, MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂ and mixtures or alloys thereof.

60. The composition according to claim 37, wherein the reflective particles comprise at least one nacre.

61. The composition according to claim 60, wherein the nacre is selected from the group consisting of nacres of golden color, bronze nacres, orange nacres, brown nacres, nacres with a coppery glint, nacres with a red glint, nacres with a yellow glint, nacres of red hue with a golden glint, pink nacres, black nacres with a golden glint, blue nacres, golden-green nacres, pink nacres, orange nacres, and mixtures thereof.

62. The composition according to claim 37, wherein the reflective particles comprise at least one goniochromatic coloring agent.

63. The composition according to claim 37, comprising at least one aqueous phase.

64. The composition according to claim 37, comprising at least one fatty phase.

65. The composition according to claim 64, said composition being anhydrous.

66. The composition according to claim 64, wherein said fatty phase contains at least one of: (a) at least one fatty substance that is liquid at room temperature and at atmospheric pressure and (b) at least one fatty substance that is solid at room temperature and at atmospheric pressure.

67. The composition according to claim 66, wherein said fatty substance that is liquid at room temperature and at atmospheric pressure comprises at least one volatile or non-volatile oil or a mixture thereof.

68. The composition according to claim 66, wherein said fatty substance that is solid at room temperature and atmospheric pressure is selected from the group consisting of waxes, pasty fatty substances, gums, and mixtures thereof.

69. The composition according to claim 37, comprising at least one additional filler.

70. The composition according to claim 37, comprising at least one film-forming polymer.

71. The composition according to claim 37, said composition being in a fluid form selected from the group consisting of: liquid, paste, direct or inverse emulsion and gel.

72. The composition according to claim 37, said composition being in a solid form selected from the group consisting of: compact, pulverulent, cast form, and in the form of a stick.

73. The composition according to claim 72, said composition being in a form selected from the group consisting of: a foundation to be applied to the face or the neck, a concealer product, a complexion corrector, a tinted cream, a makeup base for the face and a makeup composition for the body.

74. A method for making up dark skin, comprising the applying to the skin at least one composition according to claim 1.

75. A method for making up dark skin, comprising the applying to the skin at least one composition according to claim 37.

76. A kit for making up skin comprising at least one first composition and at least one second composition, each in a container, the first composition comprising, in a first physiologically acceptable medium, at least one coloring agent, and the second composition comprising, in a second physiologically acceptable medium, at least reflective particles.

77. The kit according to claim 76, wherein the coloring agent and reflective particles together provide a hue angle h ranging from 40° to 70°, and a saturation C* ranging from 20 to 50.

78. The kit according to claim 76, wherein the coloring agent and reflective particles together provide a reflectance ranging from 10% to 45% in the range of from 600 to 680 nm.

79. A method for making up dark skin, comprising applying to the skin the first composition and the second composition according to claim 76.

80. A method according to claim 79, comprising applying a first coat of one of the first and second compositions followed by applying, over at least a portion of said first coat, a second coat of the other of the first and second compositions.

81. A method for lightening a dark skin, comprising applying a composition according to claim 1 to the skin.

82. A method for lightening dark skin, comprising applying a composition according to claim 37 to the skin.

83. A method for lightening dark skin, comprising applying the first composition and the second composition according to claim 76 to the skin.

84. A composition of foundation type comprising, in a physiologically acceptable medium, at least one coloring agent and reflective particles, said composition having a hue in the color range extending from pink-beige to orange-brown.

85. A method for making up dark skin, comprising applying to the skin a first composition comprising, in a first physiologically acceptable medium, at least one coloring agent, and a second composition comprising, in a second physiologically acceptable medium, at least reflective particles.

86. The method according to claim 85, comprising applying a first coat of one of the first and second compositions followed by applying, over at least a portion of said first coat, a second coat of the other of the first and second compositions.

87. The composition according to claim 22, wherein the metallic compound is Fe₂O₃.

88. The composition according to claim 59, wherein the metallic compound is Fe₂O₃.
